(19) United States
(12) Patent Application Publication
(10) Pub. No.: US 2010/0111758 A1
(43) Pub. Date: May 6, 2010

(54) Anti-pathogenic agent delivery system

(76) Inventor: Pam Brady, Mesa, AZ (US)

Correspondence Address: Alan Carlson, 6202 Lynn Lane, Lago Vista, TX, 78645, US

(21) Appl. No.: 12/290,912

(22) Filed: Nov. 5, 2008

(51) Int. Cl. A61L 2/18

(52) U.S. Cl. 422/28; 422/292

(57) ABSTRACT

Arrangements for disinfecting appliances are disclosed. A reservoir can contain an anti-pathogenic agent and conduit having an outlet and an inlet can extend through the reservoir. The conduit can move the anti-pathogenic agent from the reservoir to outlet in response to air flow entering the inlet from a can of compressed air. In some embodiments a system is disclosed having a conduit coupled to a reservoir via a metering module all in communication with an air source where the reservoir contains an anti-pathogenic agent. Air flow from the air source can urge the anti-pathogenic agent into the conduit. Other embodiments are also disclosed.

### BACKGROUND

The present disclosure generally relates to disinfecting an object, more particularly, this disclosure relates to arrangements for an anti-pathogenic delivery system.

It has been determined that publically available computer keyboards or telephone handsets that are accessible to the public, for example at a public library, have on the average, 400 times as many microbes as a public lavatory. Apparently, this is due to the scheduled janitorial cleanings and the janitor's use of disinfectants when cleaning public restrooms. However, generally the same care and disinfecting is not performed on keyboards and telephones used by the public. Accordingly, many health organizations recommend that office equipment or appliances that have regular contact with the public be regularly disinfected to prevent the spread of viruses, bacteria, fungus, molds etc., or pathogens responsible for illnesses or diseases.

One study conducted by the University of Arizona, estimated that a desk is capable of supporting 10 million microbes and the average office contains 20,961 microbes per square inch. The study revealed that a telephone user interface which is placed proximate to one's mouth and/or ear can be home to 25,127 microbes per square inch, a computer keyboard can house 3,295 microbes per square inch and a computer mouse can harbor 1,676 microbes per square inch. By contrast, the study revealed that the average toilet seat contains 49 microbes per square inch. Microbiologist Dr. Charles Gerba, of the University of Arizona, stated, when someone is infected with or is carrying a cold or flu bug, the surfaces they touch during the day become pathogen transfer points as many cold and flu viruses survive on surfaces for as long as 72 hours.

Dr. Gerba's study also found bacteria levels increased drastically during the day, peaking after lunch because food spills support mini eco-systems, yet disinfecting user interfaces and appliances such as keyboards, telephones, etc., are not always given high priority.

In addition Dr Gerba's study found that when office workers who were told to clean their desks, keyboards, etc., with disinfecting wipes, bacterial levels were reduced on such devices by 99%. In addition, other leading experts have stated that the study reinforced the need for good hygiene practices, both at work and in the home, as our hands and things we touch are the superhighways for pathogens. Viruses, especially cold viruses, are most often transferred when an individuals hand touches a surface where harmful pathogens, particularly cold viruses are present. While it is impossible to turn our surroundings or all surfaces into sterile zones, we can minimize the risk of becoming ill by washing our hands regularly and using a disinfectant on devices we touch that have been touched by others.

### BRIEF SUMMARY

A dual purpose anti-pathogenic agent delivery system is disclosed. The system can be utilized for disinfecting user interfaces including appliances such as keyboards, telephones, a copier switch and other appliances where human hands of the public come into contact with the appliances. In some embodiments, the system can include a source of compressed air, a reservoir that contains an anti-pathogenic agent and a conduit that has an inlet and an outlet. The conduit can extend through the reservoir move the anti-pathogenic agent from the reservoir to outlet in response to user adjustable air flow entering the inlet. In addition to disinfecting, the system can be utilized to remove particles such as dust from the appliance. Such a dual purpose system can first blow pathogens from the appliance then kill any remaining pathogens with the disinfectant. As the user is releasing the compressed air, the conduit can direct the air flow in a user desired direction or a particular area of the appliance, thereby creating a force on particles proximate to the appliance. The force created by the system can be one half pound per square inch or greater depending on the source and the user settings on the valves of the system.

In some embodiments, the system has a conduit coupleable to an air source and a reservoir coupleable to the conduit, where the reservoir contains an anti-pathogenic agent. The system can also include a metering module between the conduit and the reservoir where the contents of the reservoir can be in communication with the conduit via the metering segment. Accordingly, the anti-pathogenic agent in the reservoir can be urged into the conduit in response to air from the source moving through the metering module.

In some embodiments, the reservoir can have an inner wall and an outer wall and the anti-pathogenic agent can be confined between the inner wall and the outer wall. When air released from the air source moves through the metering module, the air can remove the agent from between the inner wall and outer wall and disperse the anti-pathogenic agent to the desired area. The system can have various controls, such as valves to ensure that the proper amount of agent is dispersed for the proper application. Also, the conduit can ensure the proper amount of air is released to move particles such as food from the appliance.

For example, a first valve can control an amount of air entering the conduit and a second valve can control or regulate the amount of anti-pathogenic agent urged from the reservoir into the conduit. Another control mechanism can include an adjustable nozzle to control an amount of air exiting the conduit and the adjustable nozzle can control atomization of the agent into the air at the output of the conduit. The nozzle can also be adjusted to control a spray pattern of the air-anti-pathogenic agent mixture exiting the conduit.

In some embodiments, the metering segment can include a venturi. In addition the anti-pathogenic agent in the reservoir can be above the conduit and gravity assists to feed the anti-pathogenic agent into the metering module. In other embodiments a pressure differential can be generated in the reservoir or proximate to reservoir to assist in urging the anti-pathogenic agent into the metering module. The metering module can include a needle valve and/or the metering module can include a gate valve.

In some embodiments, the reservoir can be detachable from the metering module to make the reservoir easier to fill. In addition, two reservoirs can be utilized where an anti-pathogenic agent from the first reservoir can be mixed with an agent from the second reservoir during operation or in response to airflow. Each agent can have a specific feature, where the combination of the agents is superior to a single agent. In some embodiments, the disclosed arrangements can disperse both a solid anti-pathogenic agent and a liquid anti-pathogenic agent.

In yet another embodiment, a method for disinfecting an appliance is disclosed. The method can include assembling an anti-pathogenic agent dispersion system to a dry air source, placing an anti-pathogenic agent into a reservoir, releasing substantially dry air from the dry air source and mixing the substantially dry air with the anti-pathogenic agent. The mix can then be propelled as an anti-pathogenic agent onto a surface with the dry air source. The anti-pathogenic agent can be small solid particles suspended in a liquid, where the liquid can act as a carrier and the liquid can quickly dry leaving the solid residue behind to kill the pathogens for months without destroying any electrical or plastic components of the appliance. The dry air source can also be utilized to dry the liquid carrier and to blow pathogens carrying particles from the appliance. The dry air can defined such that no liquid droplets are visible in the air as it is being released from the system, or there is no visible “mist.”

### BRIEF DESCRIPTION OF THE SEVERAL VIEWS OF THE DRAWING

FIG. 1 is a side view of a combination duster, anti-pathogenic delivery apparatus;

FIG. 2 is an illustration of a delivery portion of the apparatus;

FIG. 3 is a depiction of another embodiment of an anti-pathogenic delivery system;

FIG. 4 illustrates combination straw/reservoir embodiment; and

FIG. 5 depicts a gravity anti-pathogen delivery system.

### DETAILED DESCRIPTION

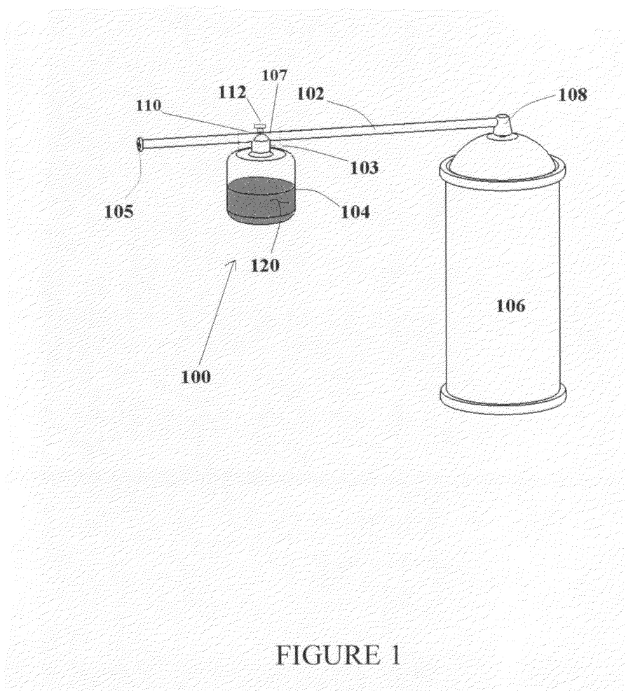

Referring to FIG. 1, an anti-pathogenic delivery system or just delivery system 100 is disclosed. The delivery system 100 can include; a compressed air source 106, conduit 102, an anti-pathogenic agent reservoir 104 that contains an anti-pathogenic agent or just agent 120, a first adjustable valve 108, a reservoir orifice 103, an adjustable spray tip 105, a venturi 110, a second adjustable valve 112 and a metering module 107, illustrated as an area between dashed lines above the reservoir 104. The first and/or second adjustable valves 108 and 112 can be a gate valves or needle valves.

The compressed air source 106 can be a can of compressed air, can be a tank connected to an air compressor, or can be a hose coming from a compressed air source 106. The air in the compressed air source 106 can be released by depressing the first adjustable valve 108. In some embodiments, the first adjustable valve 108 can be embodied as a spray tip, that when depressed releases air from the source 106 or as a trigger similar to a trigger of a firearm, where a user can activate the trigger by pulling on or pushing on the trigger with a single finger instead of pressing downward on the adjustable valve 108.

The conduit 102 can be a two part conduit, with one piece between the source 106 and the reservoir 120 and another piece between the reservoir 120 and the nozzle 105. The ends of the conduit 102 can be round and have an outside diameter that is similar in dimension to diameters of recesses in the first adjustable valve 108, the reservoir 104 and the nozzle 105. During assembly, the conduit 102 can be pressed into the first variable valve 108, the reservoir 104 and the nozzle 105. In some embodiments, the nozzle 105 can be adjusted or changed to control a spray pattern of an air anti-pathogenic agent mixture exiting the conduit 102. In other embodiments, the conduit 102, the first valve 108, the reservoir 104 and the nozzle 105 can have threaded ends/recesses that mate with each other.

In operation, air released from the source 106 can pass close to the reservoir 104 and can be in communication with the reservoir 104 and, via one or more physical phenomena such as gravity, Bernoulli principles, Boyles law, atomization etc., the agent 120 can be extracted or emptied from the reservoir 104 and dispersed onto a surface by the system 100.

Many different adjustments can be made to regulate the amount of anti-pathogenic agent 120 that is dispersed out the nozzle 105 during operation. One adjustment is the quantity of air that a user releases from the source 106 via variable valve 108. Another adjustment is the second adjustable valve 112 that can regulate the amount of air entering the reservoir 104 or amount of suction that is applied to the reservoir 104 when air is flowing in the conduit 102. The reservoir 104 can have a vent to allow air to replace the agent 120 in the reservoir 104 as the agent 120 is dispersed on the desired surface. The size of a vent on the reservoir 104 can also be adjusted to regulate the amount of agent 120 that is dispersed by the system 100.

In some embodiments, nozzle 105 can be threadably engaged with the conduit 102 and rotating the nozzle 105 can provide a greater or lesser obstruction for the air flow and the agent 120. The nozzle 105 could have a series of screens or an adjustable vortices generator. Thus, the atomization, air flow, etc., can be controlled by the nozzle 105 to provide adjustable atomization of the agent 120. Accordingly, the correct proportions of air to agent 120 and correct spray pattern can be achieved based on the setting of the nozzle 105 and the valves 108 and 112.

The metering module 107 can be couplable to the conduit and coupleable to the reservoir 104, wherein the reservoir 104 is in communication with the metering module 107 such that the anti-pathogenic agent 120 in the reservoir 104 is urged into the 102 conduit in response to air moving through the metering module 107.

The agent 120 can come in many different physical forms such as a gas, a liquid or a solid, such as a powder, or any combination thereof. Each physical form can have a variety of densities, consistencies or viscosities (DCV). The physical phenomena utilized to get the agent 120 into the conduit 120 or air stream and out the nozzle 105 can be dependent on the physical form of the agent 120 and the DVC etc., of the agent 120. The setting of the first valve 108, the second valve 112 and the nozzle 105 can be adjusted based on the physical form of the agent 120 and the DVC of the agent 120 such that a desirable distribution of the agent 120 can occur on the target surface.

The reservoir 104 can be removable and disposable or can have a fill cap and can be reusable. In other embodiments, the reservoir 104 can be removable from the conduit 102 and when the level of the agent 120 gets low, the reservoir 104 can be removed, more agent 120 can be added to the reservoir 104. Accordingly, different agents can be put in the reservoir 104 for different purposes.

As mentioned above, the system 100 can be a dual purpose system. A user can purchase a can of compressed air or an air source 106, and agent 120 and can also purchase the conduit 102. To make a disinfectant system the reservoir 104 can be filled with an agent 120 and the reservoir 120 and the conduit 102 can be assembled to the compressed air source 106. For a dusting system, the conduit 102 can be assembled to the source 104 and in some embodiments the reservoir 104 be replaced as a conduit coupler and the system can act as a duster. Thus, the source 106 can be packaged and sold as a dual purposes system as the reservoir 104 can be removed and the system 100 can act as a duster and the reservoir 104 can be added and the system 100 can act as a disinfectant system, or as an anti-pathogenic agent distribution system.

In other embodiments, parts do not have to be removed to switch modes from dusting to disinfecting. For example, when a user lightly activates variable valve 108, the agent 120 can be dispersed and when a user opens variable valve 108 wide open the system 100 can operate solely as a duster as the increased airflow and air pressure can cut off the agent flow from the reservoir 104. In other embodiments valve 112 can be adjusted such that not agent is released into the airstream.

During operation, a user can adjust the setting of valve 112 while variable valve 108 is activated and a user can visually monitor how much agent 120 the moving air is extracting from the reservoir 104. Accordingly, the user can control a quantity of agent 120 that is contained within the air stream that exits nozzle 105 by the combined adjustment of valves 108, 112 and nozzle 105. If a solid agent is utilized, the user may not want much of a dry agent, such as dust, or liquid to be dispersed. It can be appreciated that in different surface applications may utilize different agents and the amount of agent dispersed can be accurately controlled based on such valve setting features.

The nozzle 105 can function as a valve and can be considered a valve. Thus, valves 108, 112 and 105 can have calibrated settings points and a user can be provided with instructions on how to set the valves for different agents, spray patterns, items, and surfaces for desired effectiveness. It can be appreciated that some substances, such as rubbing alcohol, are very inexpensive yet very effective in killing pathogens. Thus, a user could put an inexpensive agent in the reservoir 104, properly adjust the valves 108, 112 and 105 and economically and effectively disinfect a keyboard or a telephone even in a public place. Using a small compressed air bottle such as a those utilized by whipped cream dispensers or air horns, a straw, and a reservoir the system when disassembled could be packaged in a two inch by three inch area and placed in a pocket or a purse such that the system is very mobile and can be taken to a public place.

As stated above, for the specific application, a quantity of disinfectant can be mixed with the air in the correct proportions based on the setting of the adjustable valves 108, 112 and 105. It can be appreciated that the anti-pathogenic agent used should not harm any electrical components of the target appliances.

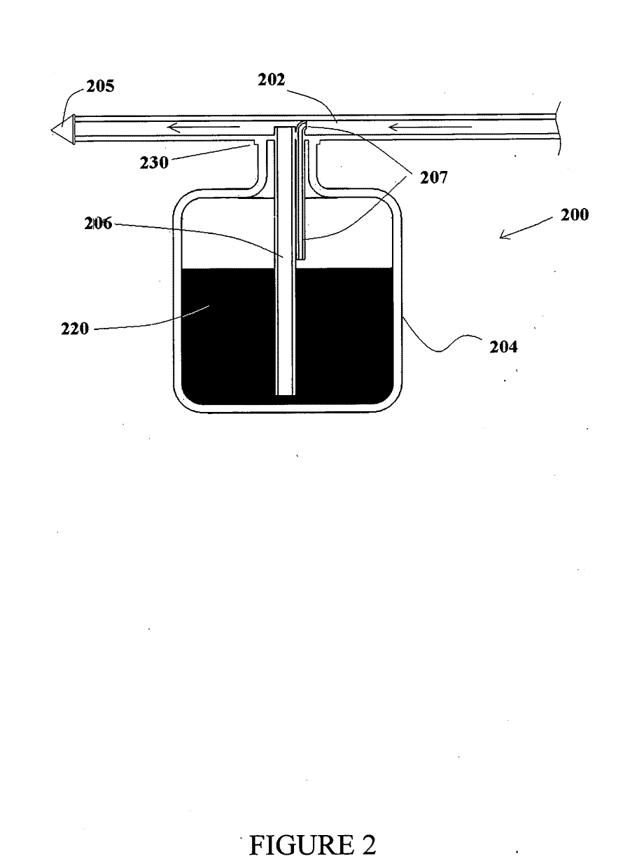

Referring to FIG. 2, a cross sectional view of an agent distribution system 200 is depicted. The system 200 can include a reservoir 204, an agent 120 contained by the reservoir 204, conduit 202, a nozzle 205, a threaded receptacle 230 for the reservoir 204, a riser tube 206 and a pressurization tube 207. The conduit 202 can direct the airflow to a desired location with a force on loose particles proximate to the location. The force can be greater than one pound per square inch. When air flow occurs through the conduit 202, the pressurization tube 207 can deflect the airflow downward, thereby pressurizing the reservoir 204. When the reservoir 204 is pressurized the agent 220 can be forced into the riser tube 206 and out the top of the riser tube 206 into the air stream and out the nozzle 205. The riser tube 206 can also reduce the cross sectional area of the conduit 202 and thus, in accordance with the Bernoulli principle, the reduced cross sectional area in the conduit can create a low pressure in the riser tube 206 and the reservoir further urging the agent 220 into the air stream.

The change in pressure inside of the reservoir can be great enough to overcome gravity as the agent 220 can be urged into the airflow in the conduit 220 such that the agent 220 can be atomized and/or dispersed as it exits the nozzle 205. The Bernoulli Effect can dictate that the static pressure in the metering area or restricted flow area of conduit 202 above the reservoir is reduced as the cross sectional area of the conduit 202 is reduced then becomes greater. With the riser tube 206 in the air flow, the higher the airspeed in the conduit 202, the higher the pressure transferred to the inside of the reservoir 204, and the larger the amount of disinfectant mixed in the airflow and dispersed by the nozzle 105. When adjusted properly, the system 200 can operate much like a carburetor, generating an accurate mixture of air and agent and atomizing such a mixture and delivering the mixture to the desired area.

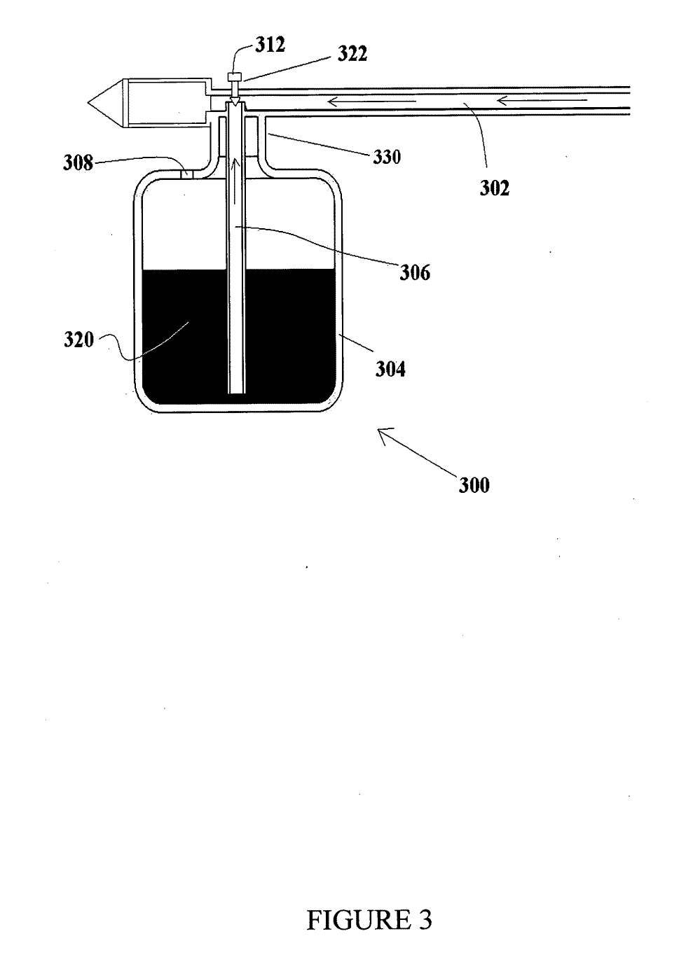

Referring to FIG. 3, an agent disbursement system 300 is depicted. The system 300 can include a reservoir 304, an agent 320, a riser tube 306, a vent 308, a venturi 322, an adjustment valve 312, a thread reservoir and a removable coupler between the reservoir 304 and the conduit 302. The adjustment valve 312 can be rotated to increase or decrease the cross sectional area of the venturi 322 above the riser tube 306. The conduit 302 that is downstream from the venture 322 can have a larger cross sectional area than the venture to create the low pressure in the reservoir 304. The reservoir 304 can engage the conduit 302 via the reservoir coupler 330 which can be a female thread. The reservoir 304 can be a male thread.

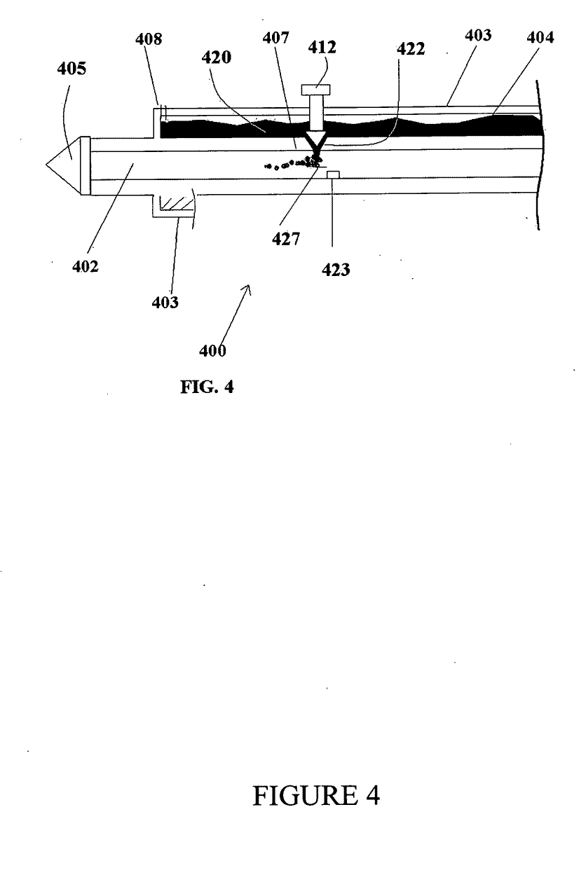

Referring to FIG. 4, an agent dispersion system 400 is illustrated. The system 400 can include a conduit 402, a reservoir 404 between the conduit 402 and an outer structure 403, an agent 420, a vent 408, a tray 427, a nozzle 405, a venturi 422, an orifice 422 between the reservoir and the conduit 402, an obstruction 423, a metering area or module 407 and an adjustable valve 412. The conduit/reservoir combination 404/404 can appear as two straws that are concentric, where the conduit 402 is surrounded by the outer structure 403 of the reservoir, where the agent 420 can be contained on the outside of the conduit 402 and inside of the outer structure 403. This embodiment is shown on the left hand side of the drawing where only a small portion of the outer structure 403 is shown as cross hatched on the bottom of the conduit 402 near or proximate to the nozzle 405 and above the majority of the length of the conduit 402.

In some embodiments, the reservoir 404 can be a tube that is tangent to the conduit 402, possibly on top of the conduit 402. Such a configuration could look like a “double barrel” shotgun. Other configurations with a varying degree of surface area attachment between the conduit 402 and the reservoir 404 could also be utilized. The conduit 402 can have an inlet and an outlet and can extend through the reservoir 404 and can move the agent 420 from the reservoir 404 to the outlet in response to air flow entering the inlet. The reservoir 404 can have an inner wall and an outer wall 403 whereby the anti-pathogenic agent can be confined between the outer wall of the conduit 202 and the outer wall 103.

The system 400 can have a venturi caused by an “obstruction” 422 where during operation a low pressure area is developed in a metering area when air flows past the obstruction 422. The low pressure area can urge the agent 420 into the conduit 402 and out the nozzle 405 with the air flow. The vent 408 can allow air to enter the reservoir 404 as the agent 420 is depleted from the reservoir 404.

The user can control the amount of air flow through the conduit 402 via a main valve of the source and the user can set valve 412. The setting of valve 412 can dictate how much agent 420 is released into the air stream and out the nozzle 405. The valve 412 can include a male thread, a female thread, a point, and a seat. Thus, in some embodiments the valve 412 can be a needle valve. In other embodiments the valve 412 can have an axel, a gate and a linkage to set the angle of the gate.

In some embodiments, the system 400 can be a disposable entity. The system can come with a can of compressed air, a single straw for dusting and a conduit/reservoir 402/404. The user can install the single straw/conduit on the can to dust and replace the single straw with the conduit/reservoir 402/404 to apply a disinfectant. The conduit/reservoir 402/424 can be two plastic straws integrated with each other and when the agent is gone from the reservoir 404, the conduit/reservoir 402/404 can be thrown away with the can that may be out of compressed air. As illustrated, the valve 412 can be set such that when the valve is adjusted properly and an opening of a specific dimension is created between the reservoir 404 to the conduit 402, a metered amount of agent 404 can dribble into the airstream in the metering area or metering module area of the conduit 404.

The adjustment valve 412 and the tray 427 can be configured like or operate like an animal feeder or animal waterer that drops an amount of agent into a tray and when the agent is removed from the tray by the air flow more water/food/agent can fall onto the tray 427 in the metering area/module 407. The metering area/module 407 can be a needle valve or a gate valve such as valve 412.

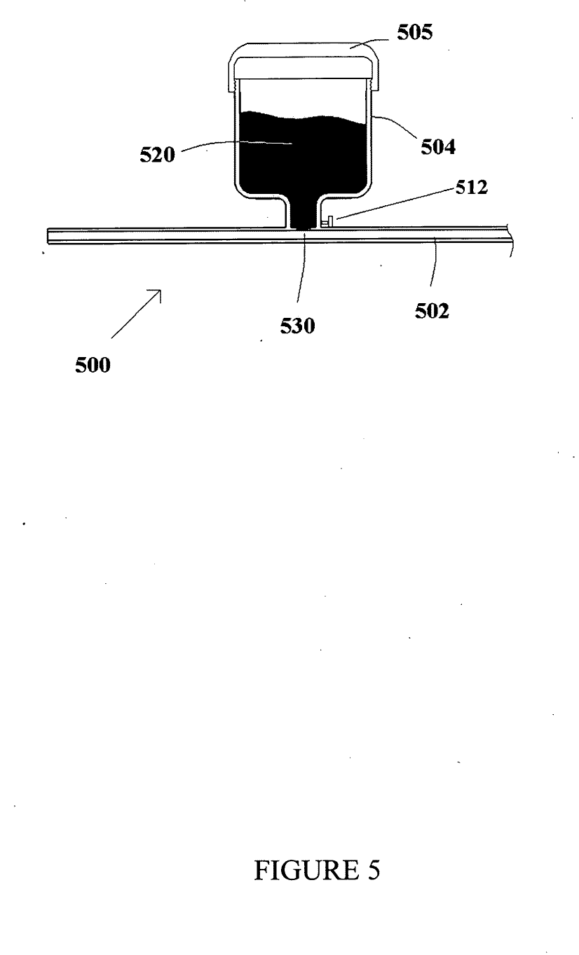

Referring to FIG. 5, a gravity feed agent dispersion system 500 is depicted. The system 500 can include a hopper or a reservoir 504, agent 520, a cap or lid 505, a conduit 502, a metering area or module 530 and an adjustment valve 512. In the disclose embodiment gravity can assist in feeding the anti-pathogenic agent 520 into a metering module 530. The system disclosed in FIG. 5 500 can be implemented when the agent 520 is in a powder form.

In some embodiments, the opening between the reservoir 504 and the conduit 502 can be adjusted such that the agent 520 will not flow down into the conduit 502 without air and or just due to gravity. Accordingly, as a drop in pressure occurs due to air flow in the metering area 530 and/or the removal of the agent from a pan or tray in the conduit 502 can urge the agent 520 into the air stream and more agent 520 can fall onto the tray. It can be appreciated that friction and surface adhesion can keep the agent 520 stable in the hopper/reservoir 504. The airflow in conduit 502 can transport particle of the agent 520 at the base of the reservoir 504 into the airstream. Accordingly, the amount of powdered agent 520 that is carried by the air stream can be proportional to the velocity and quantity of airflow and the position of the valve 512, thereby maintaining a relatively constant air to agent mixture at the nozzle 506.

It can be appreciated that with a traditional household spray disinfectant, the anti-pathogenic agent is not dispersed with sufficient force to remove particles proximate to the appliance. It can also be appreciated that with a traditional household spray disinfectant, the amount of anti-pathogenic agent dispersed is not controllable or modifiable by a user.

What is claimed is:

1. A system for disinfecting an appliance comprising:
a reservoir to contain an anti-pathogenic agent; and
conduit having an inlet and an outlet, the conduit extending through the reservoir, the conduit to move the anti-pathogenic agent from the reservoir to outlet in response to air flow entering the inlet.

2. The system of claim 1 further comprising a can of compressed air coupleable to the inlet of the conduit.

3. The system of claim 1 wherein the conduit to be utilize to direct the air flow at the appliance creating a force on particles proximate to the appliance that is greater than one pound per square inch.

4. A system comprising:
a conduit coupleable to an air source;
a reservoir coupleable to the conduit, the reservoir to contain an anti-pathogenic agent; and
a metering module couplable to the conduit and coupleable to the reservoir, wherein the reservoir is in communication with the metering segment such that the anti-pathogenic agent in the reservoir is urged into the conduit in response to air moving in the metering module.

5. The system of claim 4 wherein the reservoir has an inner wall and an outer wall and the anti-pathogenic agent is confined between the inner wall and the outer wall.

6. The system of claim 4 further comprising an air source to move air in the conduit and disperse the anti-pathogenic agent into an area proximate to an end of the conduit.

7. The system of claim 4 further comprising a first valve to control an amount of air entering the conduit.

8. The system of claim 4 further comprising a second valve to control an amount of anti-pathogenic agent urged into the conduit.

9. The system of claim 4 further comprising a nozzle to control an amount of air moved in the conduit.

10. The system of claim 4 further comprising a nozzle to control a spray pattern of an air anti-pathogenic agent mixture exiting the conduit.

11. The system of claim 4 wherein the metering segment comprises a venturi.

12. The system of claim 4 wherein the anti-pathogenic agent is above the conduit and gravity assists to feed the anti-pathogenic agent into the metering segment.

13. The system of claim 4 wherein a pressure differential is to assist in urging the anti-pathogenic agent into the metering segment.

14. The system of claim 4 wherein the metering segment comprises a needle valve.

15. The system of claim 4 wherein the metering segment comprises a gate valve.

16. The system of claim 4 wherein the reservoir is detachable from the metering segment.

17. The system of claim 4 further comprising a second reservoir and mixing a first anti-pathogenic agent from the reservoir with another agent from the second reservoir.

18. The system of claim 4 wherein the system can disperse both a solid anti-pathogenic agent and a liquid anti-pathogenic agent or a combination thereof.

19. A method of disinfecting comprising:
assembling an anti-pathogenic agent dispersion system to a dry air source;
placing an anti-pathogenic agent into a reservoir;
releasing substantially dry air from the dry air source;
mixing the substantially dry air with the anti-pathogenic agent; and
propelling the anti-pathogenic agent onto a surface with the dry air source.

20. The method of claim 19 further comprising utilizing the dry air source to blow particles from an appliance.
